SEQUENCE LISTING (5 sequences)

SEQ ID NO: 1 - DNA, 7541 bp, synthetic construct
  source 1..7541: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccgccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaattaggcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttaccaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 3 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggccagggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 4 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttccagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 5 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

(19) United States
(12) Patent Application Publication
(10) Pub. No.: US 2025/0346921 A1
(43) Pub. Date: Nov. 13, 2025

(54) AN OPTIMIZED AAV VECTOR FOR GENE THERAPY OF MUSCULAR DYSTROPHY

(71) Applicant: INDIAN INSTITUTE OF TECHNOLOGY KANPUR, KANPUR (IN)

(72) Inventors: Jayandharan Giridhara Rao, Kanpur (IN); Navaneeth Srinivasan, Kanpur (IN); Anila Varghese, Kanpur (IN); Pratiksha Sarangi, Kanpur (IN); Vijayata Singh, Kanpur (IN)

(21) Appl. No.: 18/862,617

(22) PCT Filed: Jun. 23, 2023
(86) PCT No.: PCT/IN2023/050605

§ 371 Date: Nov. 4, 2024

(30) Foreign Application Priority Data
Jun. 24, 2022 (IN) 202211036305

(51) Int. Cl. C12N 15/86; A61K 48/00

(52) U.S. Cl. CPC C12N 15/86; A61K 48/00; C12N 2750/14143; C12N 2750/14151

(57) ABSTRACT

An optimized AAV vector for gene therapy of muscular dystrophy is provided. The optimized AAV vector includes a plurality of mutant AAV9 vectors and a microdystrophin transgene (p.AAV-CBA-kozak-μDys). The optimized AAV vector demonstrate increased transduction efficiency, gene expression levels, and can potentially achieve optimal therapeutic efficacy in humans at lower vector doses. The optimized AAV vector also demonstrate improved transduction and dystrophin gene expression in a mice model of Duchenne muscular dystrophy.

### EARLIEST PRIORITY DATE

This application claims priority from a Provisional patent application filed in India having patent application No. 202211036305, filed on Jun. 24, 2022, and titled “AN OPTIMIZED AAV VECTOR FOR GENE THERAPY OF MUSCULAR DYSTROPHY” and a PCT application bearing application no. PCT/IN2023/050605, filed on Jun. 23, 2023 and titled “AN OPTIMIZED AAV VECTOR FOR GENE THERAPY OF MUSCULAR DYSTROPHY”.

### FIELD OF INVENTION

Embodiment of the present invention relates to fields of molecular biology and virology and more particularly, it relates to an optimized AAV vector for gene therapy of muscular dystrophy.

### BACKGROUND

Duchenne Muscular Dystrophy (DMD) is a monogenic X-linked disorder caused by non-sense mutations in the dystrophin gene which subjects it to degradation at a transcript level via Nonsense-mediated mRNA decay and undergoes truncated C-terminal degradation at a protein level. Dystrophin, the largest known human gene (11.5 kbp of cDNA), codes for a rod-shaped protein that connects cytoskeleton to muscle fibre cell membrane via dystrophin glycoprotein complex (DGC) thereby facilitating muscular movements. Deconinck and Dan, 2007, in their review paper, disclosed that in humans with DMD mutations, muscle fibre necrosis, inflammation, and improper electrical signal conduction are seen. This manifests as cardiomyopathy, cognitive impairment, and ambulatory issues due to overall lack of muscle functioning.

Gene therapy remains a viable option for treating DMD. Multiple serotypes of recombinant adeno-associated virus (rAAV) vectors have been extensively used for gene therapies against various monogenic diseases such as hemophilia B, spinal muscular atrophy, Leber congenital amaurosis, and for suicide gene therapies against cancers such as leukemia. Pacak et al., 2006, in their paper, disclosed that rAAV9 serotype has shown a comparatively greater skeletal and cardiomyocyte muscle cell transduction ability and hence would be ideal for targeting muscle tissues in DMD. Due to maximum packaging limit of 5 kb for rAAV virions, microdystrophin: a highly truncated product of the dystrophin gene expressed in milder dystrophies such as Becker muscular dystrophy is widely used for the DMD gene therapy applications. Gregorevic et al., 2004; Shin et al., 2011; and Bostick et al., 2011, in their papers disclosed that the rAAV microdystrophin gene therapies for DMD have demonstrated promising transduction to striated muscles, reduced cardiac fibrosis, and improved cardiac performance in mdx mice: the standard dystrophin knockout mouse model with no major immune responses.

In humans, AAV-microdystrophin constructs are being tested in multiple clinical trials (NCT02376816, NCT03368742). However, Crudele and Chamberlain, 2019, in their review paper, disclosed that only high initial vector doses of 10¹³-10¹⁴ vgs/kg have demonstrated improved outcomes in the DMD patients. Hinderer et al., 2018, and Kornegay et al., 2012, in their paper, disclosed that such high doses of the AAV vectors show inflammatory myopathy in dog pups and liver toxicity in rhesus macaque monkey models due to host innate immune responses.

In an ongoing clinical trial for DMD conducted by Solid biosciences (SGT-001), evidence of complement activation is found in a patient who suffered an adverse reaction after high dose rAAV injection (2E14 vg/kg) which led the FDA to put the trial on hold. The trial resumed following the removal of empty viral capsids produced during vector manufacturing. Recently, death of a patient is reported in a clinical trial conducted by Pfizer who was administered such high vector doses. Noris and Remuzzi, 2013, and Zaiss et al., 2008, in their paper disclosed that multiple evidence suggest that innate immune response and complement pathways are rAAV vector dose dependent.

Hence, there is a need for an optimized AAV vector for gene therapy of muscular dystrophy which demonstrate increased transduction efficiency and gene expression levels and potentially achieve optimal therapeutic efficacy in humans at lower vector doses.

### SUMMARY

In accordance with an embodiment of the present invention, an optimized AAV vector for gene therapy of muscular dystrophy is provided. The optimized AAV vector includes a plurality of mutant AAV9 vectors and a microdystrophin transgene (p.AAV-CBA-kozak-μDys). The plurality of mutant AAV9 vectors includes AAV9K51Q, AAV9N57Q and AAV9K316Q. The p.AAV-CBA-kozak-μDys includes gene sequence as set forth in SEQ ID No. 1. The AAV9K51Q includes a gene sequence as set forth in SEQ ID No. 2. The AAV9N57Q includes a gene sequence as set forth in SEQ ID No. 3. The AAV9K316Q includes a gene sequence as set forth in SEQ ID No. 4. The optimized AAV vector could exhibit improved efficiency of gene therapy in muscular dystrophy patients at lower vector doses.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of p.AAV-CBA-kozak-μDys, in accordance with an embodiment of the present invention;

FIG. 2 is a schematic representation of AAV9K51Q, in accordance with an embodiment of the present invention;

FIG. 3 is a schematic representation of AAV9N57Q, in accordance with an embodiment of the present invention;

FIG. 4 is a schematic representation of AAV9K316Q, in accordance with an embodiment of the present invention;

FIG. 5 is a schematic representation of a method for preparing an optimized adeno-associated virus (AAV) vector, in accordance with an embodiment of the present invention;

FIG. 6 is a graphical representation of in-vitro transfection of microdystrophin transgene plasmids, in accordance with an embodiment of the present invention;

FIG. 7 is a graphical representation of in-vitro transduction of AAV9WT virus packaged with microdystrophin transgene (μDys) under the control of CBA-Kozak promoter enhancer sequence and MHCK7H2 promoter sequences in HeLa and C2C12 cells, in accordance with an embodiment of the present invention;

FIG. 8 is a representation of immunofluorescence of TA muscle administered with AAV9WT vectors, in accordance with an embodiment of the present invention;

FIG. 9 is a graphical representation of in-vitro transduction of rAAV9 vectors packaged with CBA-Kozak microdystrophin in HeLa cells, in accordance with an embodiment of the present invention;

FIG. 10 is a graphical representation of in-vitro transduction of rAAV9 vectors packaged with CBA-Kozak microdystrophin in C2C12 cells, in accordance with an embodiment of the present invention;

FIG. 11 is a representation of rationally engineered AAV9 mutants (AAV9K51Q, and AAV9N57Q) in vivo in DMD mice, in accordance with an embodiment of the present invention; and

FIG. 12. is a representation of muscle strength of mdx mice after gene therapy: A grip strength meter was used to measure the phenotypic outcomes, 12 weeks post vector administration (Bioseb BIO-GS4, Vitrolles Cedex, France). A. Hind limb grip strength of mice treated with AAV9WT-CBA-Koz-μDys (n=8), AAV9K51Q-CBA-Koz-μDys (n=7) and AAV9N57Q-CBA-Koz-μDys (n=8) vectors is significantly higher than mock treated animals (n=8). AAV9K51Q-CBA-Koz-μDys treated mice had a significantly higher grip strength than AAV9WT-CAG-Koz-μDys treated mice. B. The grip strength of all the four limbs of mice treated with AAV9WT-CBA-Koz-μDys (n=8), AAV9K51Q-CBA-Koz-μDys (n=7) and AAV9N57Q-CBA-Koz-μDys (n=8) vectors is significantly higher than mock treated animals (n=8) (1B). Each data point represents the average value of 5 readings measured per animal. The data obtained is represented as mean±S.D. Statistical analysis was performed by one-tailed unpaired student t-test using GraphPad Prism V8.0.2. p-values<0.05 were considered to be statistically significant for each comparison. *p<0.05, **p<0.01, ***p<0.001, ****p<0.0001, in accordance with an embodiment of the present invention.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present invention relates to an optimized AAV vector for gene therapy of muscular dystrophy. The invention mainly focuses on development of bioengineered AAV9 vectors and kozak driven dystrophin transgene for gene delivery in Duchenne muscular dystrophy (DMD).

As used herein the term “AAV vector” refers to replication-defective, single-stranded DNA parvovirus that require a helper Ad for their replication.

In an embodiment of the present invention, an optimized AAV vector for gene therapy of muscular dystrophy is provided. The optimized AAV vector for gene therapy of muscular dystrophy comprises a plurality of mutant AAV9 vectors and a microdystrophin transgene, p.AAV-CBA-kozak-μDys, having a gene sequence as set forth in SEQ ID No. 1. The plurality of mutant AAV9 vectors consisting of AAV9K51Q, AAV9N57Q, and AAV9K316Q. The AAV9K51Q is a Neddylation mutant. The AAV9K51Q is having a gene sequence as set forth in SEQ ID No. 2. The AAV9N57Q is having a gene sequence as set forth in SEQ ID No. 3, and the AAV9K316Q is having a gene sequence as set forth in SEQ ID No. The optimized AAV vector is configured to administer to humans through one of intramuscular route, and intravenous administration at lower vector doses of 1-2×10¹¹ vgs/leg.

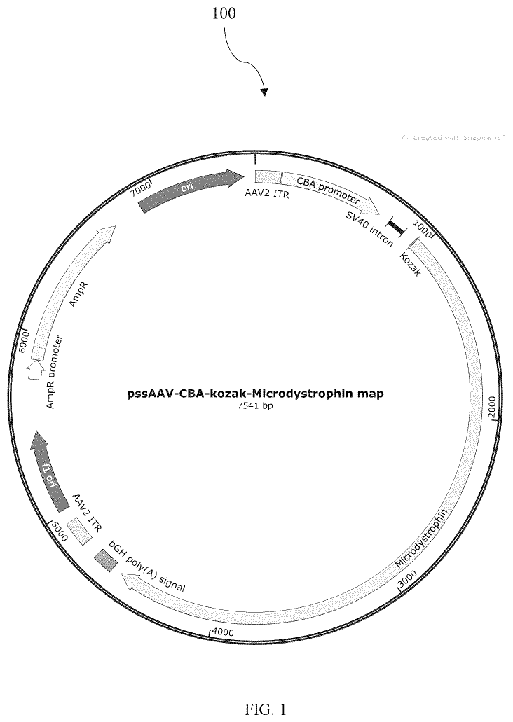

FIG. 1 is a schematic representation of p.AAV-CBA-kozak-μDys, in accordance with an embodiment of the present invention.

The optimized AAV vector including p.AAV-CBA-kozak-μDys sequence is provided in SEQ ID No. 1.

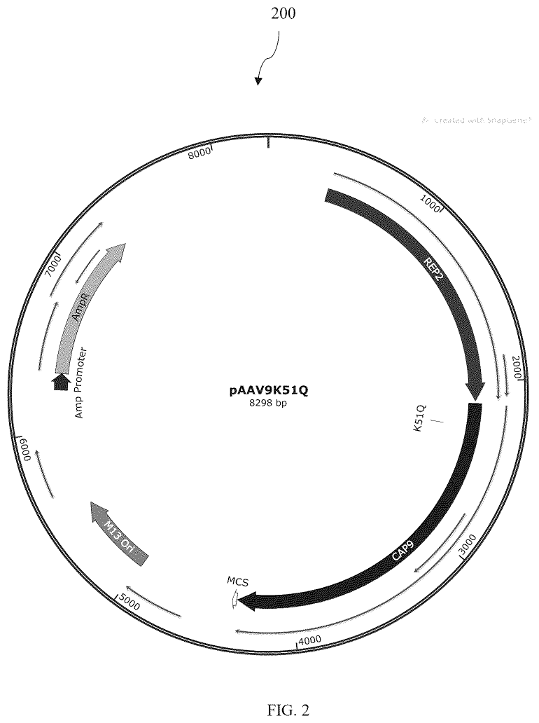

FIG. 2 is a schematic representation of AAV9K51Q, in accordance with an embodiment of the present invention.

The plurality of mutant AAV9 vectors including AAV9K51Q (Neddylation mutant) sequence is provided in SEQ ID No. 2.

### Key:

Site directed mutagenesis at codon 51 (nucleotide 2284-2286) from AAA→CAA encodes for glutamine (Q).

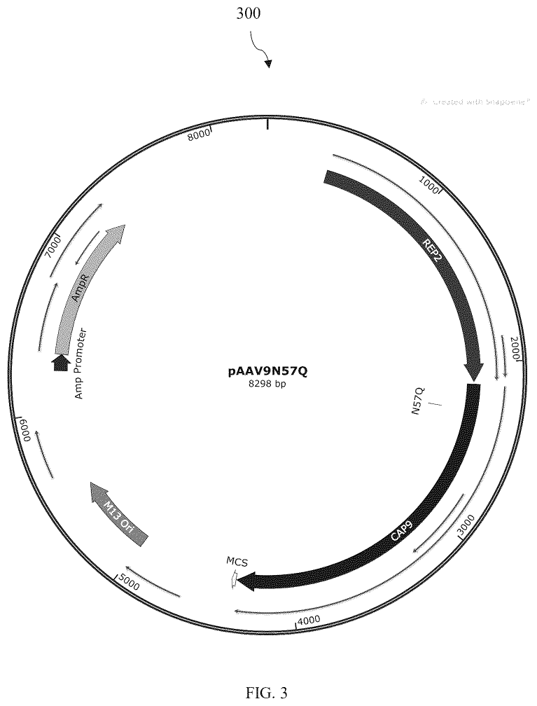

FIG. 3 is a schematic representation of AAV9N57Q, in accordance with an embodiment of the present invention.

The plurality of mutant AAV9 vectors including AAV9N57Q sequence is provided in SEQ ID No. 3.

### Key:

Site directed mutagenesis at codon 57 (nucleotide 2302-2304) from AAC→CAG encodes for glutamine (Q).

The plurality of mutant AAV9 vectors including AAV9K316Q sequence is provided in SEQ ID No. 4.

### Key:

Site directed mutagenesis at codon 316 (nucleotide 3079-3081) from AAG→CAG encodes for glutamine (Q).

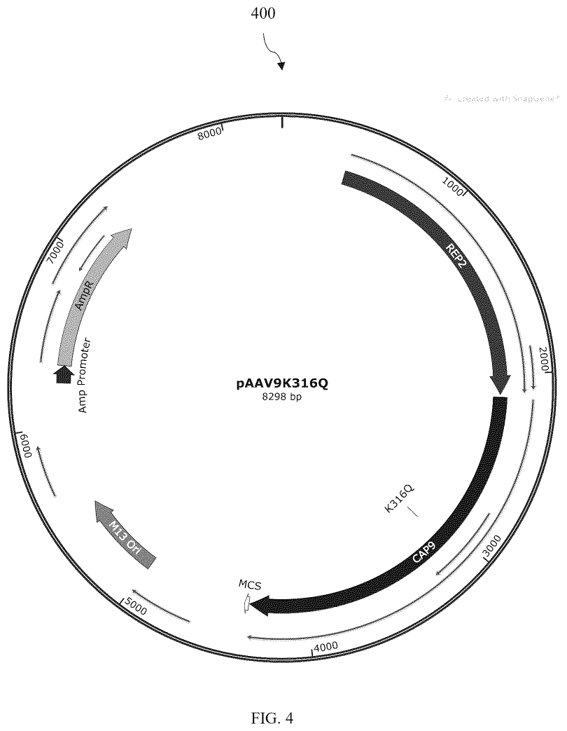

FIG. 4 is a schematic representation of AAV9K316Q, in accordance with an embodiment of the present invention.

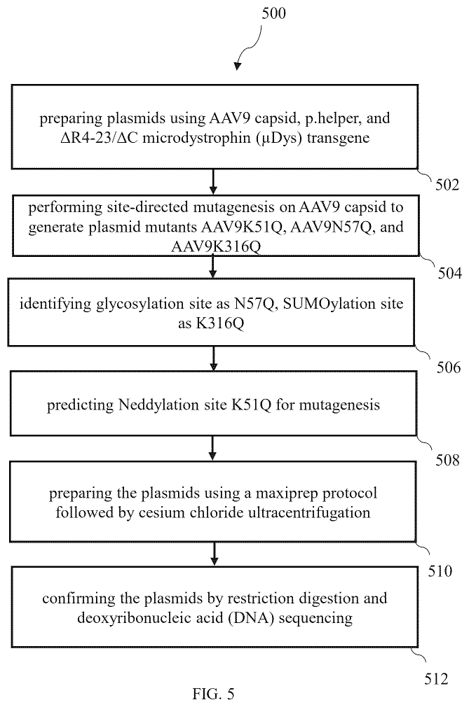

FIG. 5 is a schematic representation of a method for preparing an optimized adeno-associated virus (AAV) vector, in accordance with an embodiment of the present invention.

In another embodiment of the present invention, a method for preparing an optimized adeno-associated virus (AAV) vector is provided. The method for preparing an optimized adeno-associated virus (AAV) vector comprising the steps of preparing plasmids using AAV9 capsid, p.helper, and ΔR4-23/ΔC microdystrophin (μDys) transgene, at step 502. At step 504, the site-directed mutagenesis is performed on AAV9 capsid to generate plasmid mutants AAV9K51Q, AAV9N57Q, and AAV9K316Q. At step 506, glycosylation site is identified as N57Q, SUMOylation site as K316Q. At step 508, Neddylation site is predicted as K51Q for mutagenesis. At step 510, the plasmids are prepared using a maxiprep protocol followed by cesium chloride ultracentrifugation. At step 512, the plasmids are confirmed by restriction digestion and deoxyribonucleic acid (DNA) sequencing.

In another embodiment of the present invention, a method for preparing an optimized adeno-associated virus (AAV) vector comprises the steps of synthesizing a microdystrophin transgene, pssAAV-CBA-Kozak-μDys and pssAAVMHCK7H2-μDys is used as a transgene control. The AAV 293 cells are maintained in Iscove's-modified Dulbecco's medium (IMDM) supplemented with 10% FBS, piperacillin and ciprofloxacin. The AAV 293 cells are co-transfected with three plasmids including p.helper, one of AAV9 (rep/cap) wild type or the plasmid mutants AAV9K51Q, AAV9N57Q, and AAV9K316Q, and with the microdystrophin transgene (p.AAV-CBA-kozak-μDys) using polyethyleneimine. The medium is replaced with complete IMDM 6 hrs post-transfection. The cells are scrapped 72 hrs post-transfection followed by storing at −80° C. till further processing. The cells are lysed followed by digesting with Benzonase to obtain a virus. The virus is purified by iodixanol gradient ultracentrifugation and ion exchange chromatography. The virus titers are determined by quantitative PCR using ATCC as standards. The AAV 293 cells are sub-cultured after treatment with trypsin, washed, and re-suspended in complete medium. The AAV9-WT capsid is configured as a control in packaging the transgene control plasmid p.AAV MHCK7H2-μDys.

In another embodiment of the present invention, a composition for gene therapy of muscular dystrophy, comprising the optimized AAV vector as claimed in claim 1, in combination with a pharmaceutically acceptable carrier. The composition is administered to mdx mice through one of intramuscular route, and intravenous administration at lower vector doses of 1-2×10¹¹ vgs/leg. The composition is configured for increased microdystrophin expression and restoration of dystrophin glycoprotein complex proteins for improving muscle function.

In the present invention methods for preparing and characterizing the optimized AAV vector are provided as follows:

### 1. Plasmids:

AAV9 capsid, p.helper and ΔR4-23/ΔC microdystrophin (μDys) transgene are used for this study. The pssAAV-CBA-Kozak-μDys is synthesized (Genscript, NJ, USA) while a control pssAAVMHCK7H2-μDys (from Dr. Jeffrey Chamberlain, University of Washington) are used. Site directed mutagenesis on AAV9 capsid is performed to generate the following plasmids: AAV9K51Q, AAV9N57Q and AAV9K316Q. Of these the glycosylation [N57Q], SUMOylation [K316Q] are identified by experimental LC-MS analysis while the Neddylation site [K51Q] is predicted (NeddyPreddy) for further mutagenesis. The plasmids are prepared using maxiprep protocol followed by cesium chloride ultracentrifugation. They are confirmed by restriction digestion and DNA sequencing.

### 2. Virus Preparation:

AAV 293 cells are maintained in Iscove's-modified Dulbecco's medium (IMDM) supplemented with 10% FBS, piperacillin and ciprofloxacin. Cells are grown as adherent cultures in incubators maintained at 37° C. and 5% CO₂. Cells are sub-cultured after treatment with trypsin for 2-5 minutes at room temperature, washed and re-suspended in complete medium. AAV 293 cells are co-transfected with three plasmids; p.helper, AAV9 (rep/cap) wild type or with the mutant AAV9 [AAV9K51Q, AAV9N57Q and AAV9K316Q] and with the microdystrophin transgene (p.AAV-CBA-kozak-μDys) using polyethyleneimine. The transgene control (p.AAV MHCK7H2-μDys) is packaged only with AAV9-WT capsid to serve as the control. The medium is replaced by complete IMDM, six hours post-transfection. Cells were scraped 72 hours post-transfection and stored at −80° C. till further processing. Cells are lysed by 3 rounds of freeze-thaw and digested with Benzonase. Virus is purified by iodixanol gradient ultra-centrifugation followed by ion exchange chromatography using HiTrap Q column and concentrated by centrifugation using Amicon centrifugal spin concentrators. Titres of the virus in vgs/ml are determined by qPCR using ATCC as standards.

The transgene control (p.AAV MHCK7H2-μDys) packaged with AAV9-WT capsid sequence (pAAV9rep/cap) is provided in SEQ ID No. 5.

### Key:

Nucleotide 378-2117-Rep2 gene
Nucleotide 2134-4344-CAP9 gene

### 3. In-Vitro Transfection Assay:

HeLa cells and C2C12 cells are seeded in 24 well plate at density of 30000 cells per well. Cells are allowed to adhere by leaving it overnight in an incubator maintained at 37° C. and 5% CO₂. 500 ng of plasmids, pssAAV-CBA-Kozak-μDys & pssAAVMHCK7H2-μDys, are added to each well using polyethelenimine (PEI) as transfecting agent (PEI:Plasmid-3:1) in IMDM. Six hours after transfection, IMDM is replaced with complete IMDM. Forty-eight hours later cells are collected by adding TRIzol. RNA is isolated by isopropanol and ethanol precipitation. About 1 μg total RNA is converted to cDNA using the cDNA synthesis kit. Microdystrophin expression is analysed by quantitative reverse transcriptase PCR and the expression is normalised with respect to 18S rRNA.

Table 1 enlists sequences of the primers for site directed mutagenesis.

TABLE 1

| Virus - Site of | |
|---|---|
| mutagenesis | Primer |
| AAV9-K51Q-FP | cttgtgcttccgggttaccaataccttggacccggcaacgg |
| AAV9-K51Q-RP | ccgttgccgggtccaaggtattggtaacccggaagcacaag |
| AAV9-N57Q-FP | caaataccttggacccggccagggactcgacaagggggagccggtc |
| AAV9-N57Q-RP | gaccggctcccccttgtcgagtccctggccgggtccaaggtatttg |
| AAV9-K316Q- | ggcctaagcgactcaacttccagctcttcaacattcaggt |
| FP | |
| AAV9-K316Q- | acctgaatgttgaagagctggaagttgagtcgcttaggcc |
| RP | |

Key:
The nucleotides in the primers highlighted by bold font are the modified sequences for performing respective site directed mutagenesis.
FP - Forward primer
RP - Reverse primer.

### 4. In-Vitro Transduction Assay:

HeLa cells and C2C12 cells are seeded in 24 well plate at density of 30000 cells per well. Cells are allowed to adhere by leaving it overnight in an incubator maintained at 37° C. and 5% CO₂. Cells are transduced at an MOI of 100000 with AAV9WT-CBA-Kozak-μDys/MHCK7H2-μDys and AAV9-WT-CBA-Kozak-μDys mutants with AAV9-scEFGP as positive control for transduction. Three hours after transduction, IMDM is replaced with complete IMDM. 48 hours after transduction cells are collected by adding TRIzol. RNA is isolated by isopropanol and ethanol precipitation and 1 μg total RNA is converted to cDNA using the cDNA synthesis kit. Microdystrophin expression is analysed by quantitative reverse transcriptase PCR and the expression is normalised with respect to 18srRNA.

### 5. In-Vivo Administration:

The recombinant vector is administered to 7-16 weeks old mdx^{4cv} mice intramuscularly. AAV9WT-CBA-Kozak-μDys and AAV9WT-MHCK7H2-μDys are injected at a dose of 3.42×10¹¹ vgs/leg on the tibialis anterior (TA) muscle at a constant volume of 20 μl per leg (n=8 per group). To evaluate the performance of mutant vectors in-vivo, AAV9 WT and mutant AAV9-CBA-Kozak-μDys vectors are injected at a dose of 1×10¹¹ vgs/leg in TA muscle (n=6 per group). The mice are anesthetized and the TA muscle is exposed by an incision. The vector is administered using a Hamilton syringe. The incision is sutured following injection. Functional assays are carried out 8-10 weeks and 3 months after the administration of the vectors.

### Immunofluorescence:

TA muscles are excised from C57BL6 and mdx^{4cv} mice. Excess moisture from the muscle is removed. The muscle sample is snap frozen for 20 seconds using liquid nitrogen chilled isopentane. The frozen sample is kept on dry ice for 20 minutes to evaporate the isopentane and stored in −80° C. until sectioning. 6 μm thick sections are cut at −25° C. in a cryotome. Sections are transferred to Poly-L-lysine coated super frost slides and stored at −20° C. till staining. The sections are fixed in 4% paraformaldehyde for 20 minutes followed by washing in PBS for 10 minutes with gentle agitation at room temperature. Sections are incubated in a blocking buffer (PBS, 10% goat serum, 0.1% Triton-100) at 4° C., overnight. Sections are immunostained for Dystrophin (1:50) Dystrobrevin (1:200), α-Syntrophin (1:200), β-Dystroglycan (1:50) and nNOS (1:100), incubated in moist chamber at 4° C., overnight. Sections are washed three times for 10 minutes in PBS and incubated with secondary antibodies Alexa Fluor 568 goat anti mouse (1:250) and goat anti rabbit Cy3 (1:100) at 4° C., overnight. Sections are counterstained with DAPI for 5-10 seconds and washed in PBS 3 times for five minutes each. The slides are mounted with fluor save and imaged in a fluorescence/confocal microscope.

### Functional Assays:

Three months post vector administration, the muscle strength of mock treated and vector treated mdx mice of different experimental groups were assessed as described earlier (Aartsma-Rus and van Putten 2014).

The muscular strength of hind limbs of untreated and vector treated DMD mice was measured using a computerized grip strength meter (Bioseb, BIO-GS4, Vitrolles Cedex,France) (Montilla-García, et al., 2017). Briefly the mice were scruffed and were allowed to grab the mesh wire with their fore limbs, and the hind limbs of the mice was allowed to grasp the T-rod attached to the grip strength meter. Once the mice grabbed the T rod using hind limbs, the animal was pulled gently using the tail until hindlimbs were released from the T rod and the peak grip strength force in newtons was recorded. Peak grip strength was measured for 5 times for each animal and the mean force recorded in Newton (N) (Mucha, et al., 2021). Similarly, to assess the grip strength for all the four limbs, the mice was placed on the grid attached to grip strength meter, after it grasped the grid with all its four limbs, the animal was pulled gently using its tail until it released the grid (Mandillo et al., 2008). For each mouse, five peak grip strength readings were taken in Newton and the average was computed.

### Results and Discussion:

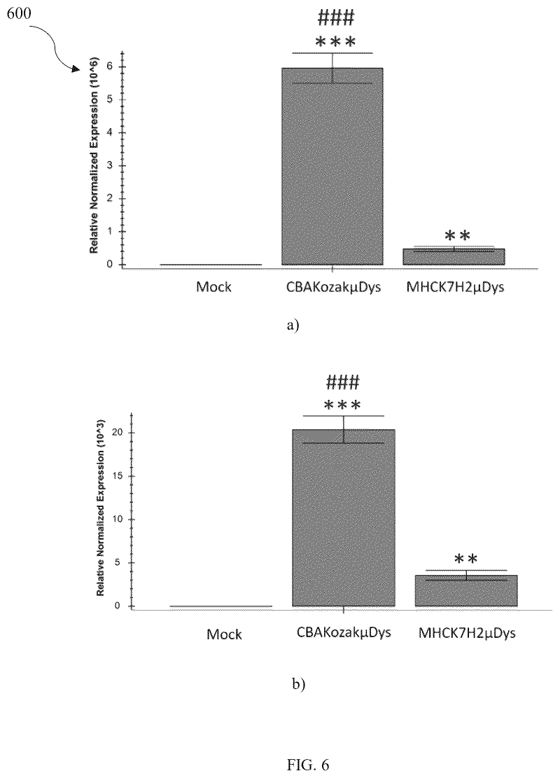

FIG. 6 is a graphical representation of in-vitro transfection of microdystrophin transgene plasmids, in accordance with an embodiment of the present invention. Microdystrophin under the control of CBA promoter-Kozak sequence (CBAKozakμDys) showed better mRNA expression compared to MHCK7H2 promoter sequences (MHCK7H2μDys) in HeLa cells (a) and C2C12 cells (b). * Represents statistical comparisons between mock treated cells and corresponding plasmid and #refers to statistical comparison between MHCK7H2 μDys and corresponding plasmid. **−p≤0.01, ***−p≤0.001, ###−p≤0.001, in accordance with an embodiment of the present invention.

### 1. Validation of CBA-Kozak Sequence Driving Expression of the Microdystrophin Transgene:

The initial goal is to validate a microdystrophin transgene construct for DMD gene therapy whose expression is driven by a hybrid CBA promoter/enhancer and a novel Kozak sequence (Henceforth referred to as CBA-Kozak sequence). The chimeric promoter/enhancer sequence consists of a cytomegalovirus (CMV) enhancer and chicken-β-actin (CBA) promoter sequences utilised in combination with novel Kozak sequence, a consensus ribosome binding site for ubiquitous and robust gene expression. The expression profile of this construct is compared with a similar microdystrophin construct driven by MHCK7H2 promoter/enhancer sequence that is currently used in clinical trials. Towards this purpose, the transgene constructs are tested in-vitro for their gene expression levels in HeLa cells, a human cervical cancer cell line and C2C12 cells, a murine myoblast cell line. Comparison of steady state mRNA levels of ΔR4-23/ΔC microdystrophin construct driven by CBA-kozak and MHCK7H2 sequences following transfection revealed that the CBA-kozak construct had a twelvefold and fivefold higher expression in HeLa and C2C12 cells, respectively compared to the MHCK7H2 construct (FIG. 6).

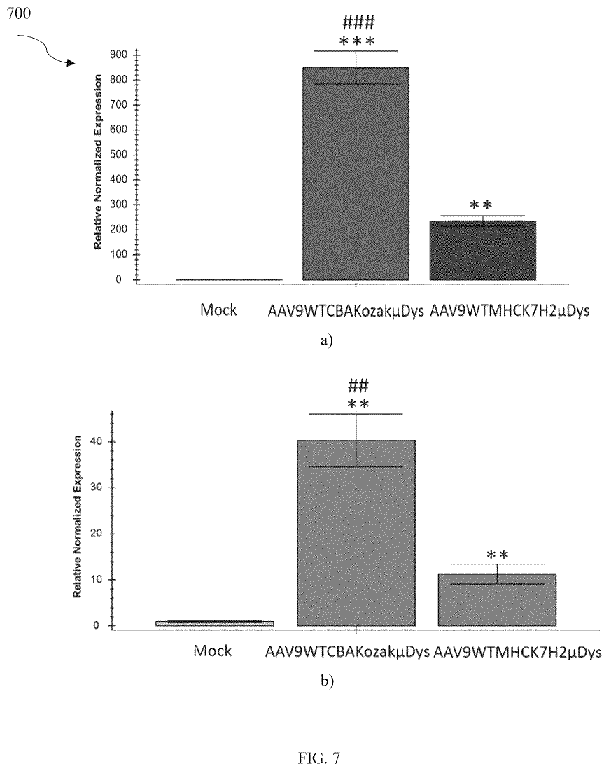

FIG. 7 is a graphical representation of in-vitro transduction of AAV9WT virus packaged with microdystrophin transgene (μDys) under the control of CBA-Kozak promoter enhancer sequence and MHCK7H2 promoter sequences in HeLa and C2C12 cells, in accordance with an embodiment of the present invention. Transduction assay revealed an increased expression of μDys driven by CBA-Kozak sequence (AAV9WTCBAKozakμDys) in cell lines of human origin (HeLa, A) and murine myoblasts (C2C12, B) compared to MHCK7H2 driven μDys (AAV9WTMHCK7H2μDys), a construct that is currently used in multiple clinical trials. * Represents statistical comparisons between mock treated cells and corresponding virus and #refers to statistical statistical comparison between AAV9WTMHCK7H2 μDys. **−p≤0.01, ***−p<0.001, ##−p≤0.01, ###−p≤ 0.001, in accordance with an embodiment of the present invention.

Transgene constructs are packaged in AAV9WT capsids to measure their in-vitro gene expression. Transduction of AAV9WT-CBA-Kozak-μDys and AAV9WT-MHCK7H2-μDys in HeLa and C2C12 cells also demonstrated relatively higher expression levels of the CBA-Kozak-μDys construct compared to MHCK7H2 microdystrophin construct (FIG. 7).

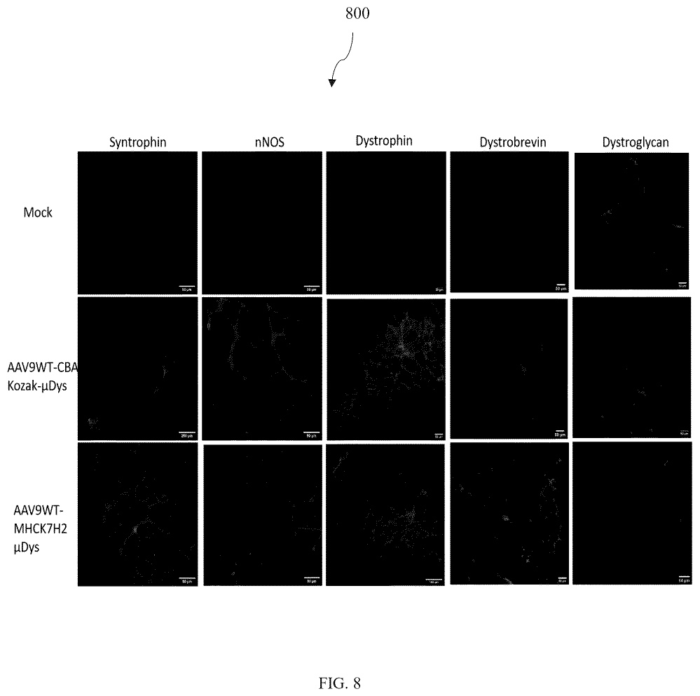

FIG. 8 is a representation of immunofluorescence of TA muscle administered with AAV9WT vectors, in accordance with an embodiment of the present invention. Restoration of dystrophin and dystrophin glycoprotein complex proteins at the sarcolemmal membrane of TA muscles of mdx mice revealed by immunofluorescence staining following intramuscular administration, in accordance with an embodiment of the present invention.

These viruses are administered to mdx mice through intramuscular route. At 8 weeks of intervention, Immunofluorescence of TA muscle cryosections from treated mdx mice revealed the expression of microdystrophin driven by both CBA-Kozak and MHCK7H2 expression cassette sequences. Correspondingly, the rescue of other dystrophin glycoprotein complex (DGC) proteins is also observed following restoration of dystrophin expression. The DGC proteins are found to be localized to the sarcolemmal membrane (FIG. 8).

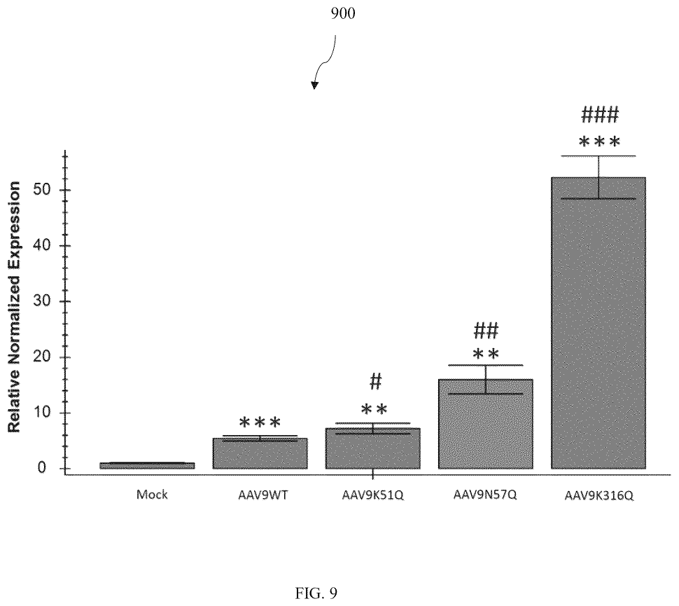

FIG. 9 is a graphical representation of in-vitro transduction of rAAV9 vectors packaged with CBA-Kozak microdystrophin in HeLa cells, in accordance with an embodiment of the present invention. The AAV9K316Q, a vector mutant for SUMOylation PTM is shown to have the highest in-vitro transduction efficiency. All the rationally engineered mutants are found to transduce better compared to AAV9WT vectors. * Represents statistical comparisons between mock treated cells and corresponding virus and #refers to statistical comparison between AAV9WTCBAKozak μDys and corresponding virus. **−p≤0.01, ***−p≤0.001, #−p≤0.05, ##−p≤0.01, ###−p≤0.001, in accordance with an embodiment of the present invention.

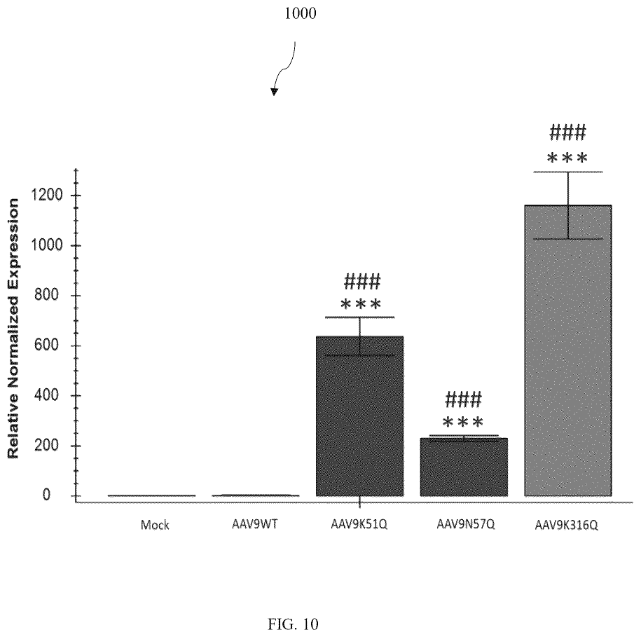

FIG. 10 is a graphical representation of in-vitro transduction of rAAV9 vectors packaged with CBA-Kozak microdystrophin in C2C12 cells, in accordance with an embodiment of the present invention. The AAV9K316Q, a vector mutant for SUMOylation PTM is shown to have the highest in-vitro transduction efficiency in the mouse muscle cell line followed by Neddylation mutant, the AAV9K51Q and the AAV9N57Q mutant for glycosylation modification. All the rationally engineered mutants are found to transduce better compared to AAV9WT vectors. * Represents statistical comparisons between mock treated cells and corresponding virus and #refers to statistical comparison between AAV9WTCBAKoz μDys and corresponding virus. ***−p≤0.001, ###−p≤0.001, in accordance with an embodiment of the present invention.

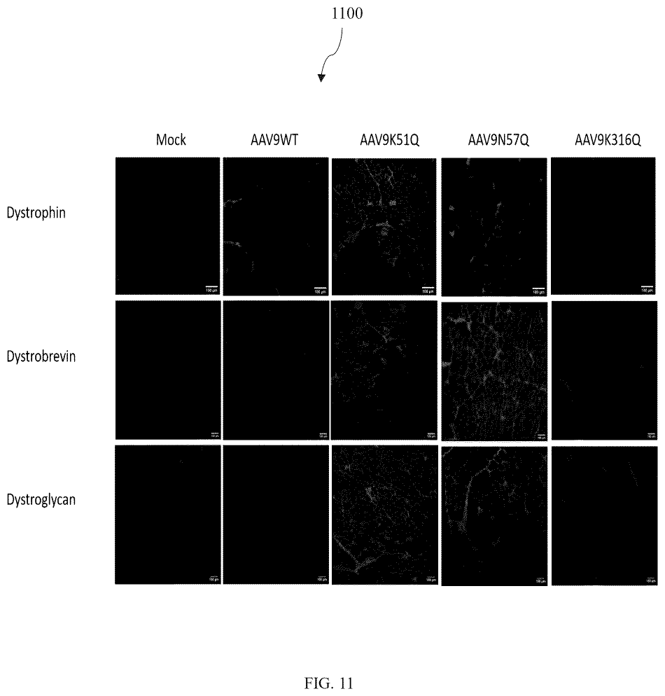

FIG. 11 is a representation of rationally engineered AAV9 mutants (AAV9K51Q, and AAV9N57Q), in accordance with an embodiment of the present invention. The AAV9K51Q and AAV9N57Q have improved expression of dystrophin glycoprotein complex (DGC) proteins at the sarcolemmal membrane compared to WT vectors as shown by TA muscle immunofluorescence, in accordance with an embodiment of the present invention.

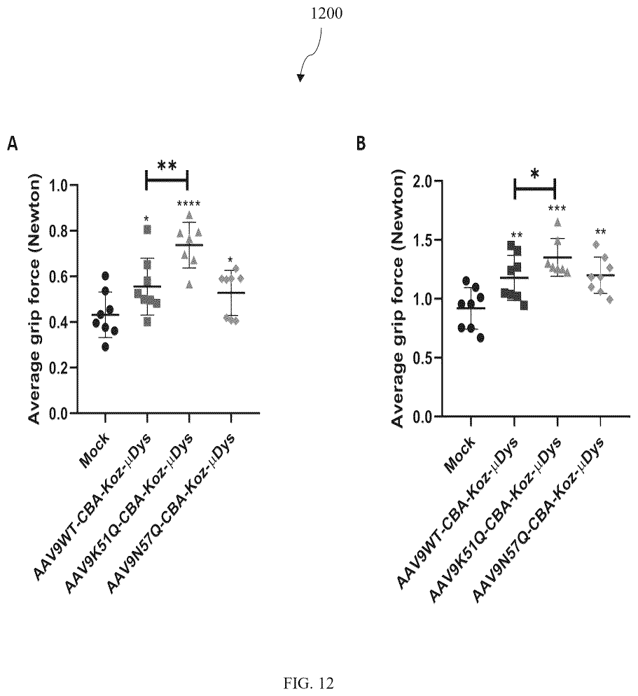

FIG. 12. is a representation of muscle strength of mdx mice after gene therapy: A grip strength meter (Bioseb BIO-GS4, Vitrolles Cedex, France) was used to measure the phenotypic outcomes, 12 weeks post vector administration. A. Hind limb grip strength of mice treated with AAV9WT-CBA-Koz-μDys (n=8), AAV9K51Q-CBA-Koz-μDys (n=7) and AAV9N57Q-CBA-Koz-μDys (n=8) vectors is significantly higher than mock treated animals (n=8). AAV9K51Q-CBA-Koz-μDys treated mice had a significantly higher grip strength than AAV9WT-CBA-Koz-μDys treated mice. B. The grip strength of all the four limbs of mice treated with AAV9WT-CBA-Koz-μDys (n=8), AAV9K51Q-CBA-Koz-μDys (n=7) and AAV9N57Q-CBA-Koz-μDys (n=8) vectors is significantly higher than mock treated animals (n=8) (1B). Each data point represents the average value of 5 readings measured per animal. The data obtained is represented as mean±S.D. Statistical analysis was performed by one-tailed unpaired student t-test using GraphPad Prism V8.0.2. p-values<0.05 were considered to be statistically significant for each comparison. *p<0.05, **p<0.01, ***p<0.001, ****p<0.0001, in accordance with an embodiment of the present invention.

### 2. Comparison of AAV9WT and Mutants:

To determine the transduction efficiency of novel AAV9 capsid mutants, in-vitro transduction assay is performed in HeLa and C2C12 cells. In both cell lines, the mutant AAV9 vectors performed better than AAV9WT vectors indicating that the post translational modifications negatively regulate transduction of AAV9. In HeLa cells (FIG. 9), AAV9K316Q-a SUMOylation mutant had the best transduction (50× higher) than that of the AAV9-WT vector. In murine muscle derived cell line C2C12 (FIG. 10), AAV9K316Q had a superior transduction efficiency almost 1000 times greater than AAV9-WT vector. The other rationally engineered rAAV9 mutants also had better transduction efficiency compared to WT vectors in both the cell lines.

These vectors are tested for their therapeutic utility by intramuscular (TA muscle) administration in mdx mice. Ten weeks post vector administration, the cryosections of muscle samples are taken and analyzed for DGC protein expression. The rationally engineered rAAV9 vectors, AAV9K51Q and AAV9N57Q had better transduction efficiency in muscle cells in comparison with their AAV9-WT counterpart as revealed by the increase in dystrophin positive muscle fibres (FIG. 11). This correlated with an improved expression of other DGC proteins, Dystrobrevin and dystroglycan, at the muscle fibre membrane. Thus, the rationally engineered rAAV9 vectors have been shown to achieve increased gene transfer through localized injection in TA muscle and could be expected to improve muscle functioning through a whole-body systemic administration.

The in vivo muscle strength of mice was measured in a non-invasive way using grip strength meter which is a very sensitive method to measure the grip force. For hind limb grip strength (FIG. 11A), the treated group showed generally higher mean grip strength (Mean 0.56 N-0.74 N Vs 0.43 N) when compared to the mock treated animals. A significant increase in hind limbs grip strength was also observed in AAV9K51Q-CBA-Koz-μDys vector treated group compared to the group treated with the AAV9WT-CBA-Koz-μDys vector (Mean 0.74 N and 0.56 N respectively, p<0.01). A higher muscle grip strength (Mean 1.18 N-1.35 Vs 0.92 N) was also observed in the four limbs grip strength assessment in all the treated groups when compared to mock group (FIG. 11B). Moreover, mice treated with AAV9K51Q-CBA-Koz-μDys vector had a relatively higher grip strength compared to the mice treated with AAV9WT-CBA-Koz-μDys (Mean 1.35 N and 1.18 N respectively, p<0.05). This demonstrates that micro-dystrophin gene therapy with AAV9K51Q-CBA-Koz-Dys vector can rescue muscle function more effectively than AAV9WT-CBA-Koz-Dys vector.

The present invention provides the optimized AAV vector for gene therapy of muscular dystrophy. The present invention exhibit improvement of microdystrophin protein expression levels by introduction of a kozak ribosome binding site to a ubiquitous chicken β-actin promoter to drive gene expression. The present invention involves targeting post translational modification sites of AAV capsid to improve their host cell transduction. The optimized AAV vectors undergo host cell-mediated post-translational modifications (PTMs) such as glycosylation and ubiquitination like modifications (UBLs) such as SUMOylation and Neddylation on viral capsids. The optimized AAV vector demonstrate increased transduction efficiency, gene expression levels, and potentially achieve optimal therapeutic efficacy in humans at lower vector doses. The optimized AAV vector also demonstrate improved transduction and dystrophin gene expression in mice model of Duchenne muscular dystrophy.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. An optimized AAV vector for gene therapy of muscular dystrophy, comprising:
a plurality of mutant AAV9 vectors,
wherein the plurality of mutant AAV9 vectors consisting of AAV9K51Q, AAV9N57Q, and AAV9K316Q,
wherein the AAV9K51Q is a Neddylation mutant,
wherein the AAV9K51Q is having a gene sequence as set forth in SEQ ID No. 2,
the AAV9N57Q is having a gene sequence as set forth in SEQ ID No. 3, and
the AAV9K316Q is having a gene sequence as set forth in SEQ ID No. 4; and
a microdystrophin transgene, p.AAV-CBA-kozak-μDys, having a gene sequence as set forth in SEQ ID No. 1.

2. The optimized AAV vector for gene therapy of muscular dystrophy as claimed in claim 1, wherein the optimized AAV vector is configured to administer to humans through one of intramuscular route, and intravenous administration at lower vector doses of 1-2×10¹¹ vgs/leg.

3. A method for preparing an optimized adeno-associated virus (AAV) vector, comprising the steps of:
preparing plasmids using AAV9 capsid, p.helper, and ΔR4-23/ΔC microdystrophin (μDys) transgene;
performing site-directed mutagenesis on AAV9 capsid to generate plasmid mutants AAV9K51Q, AAV9N57Q, and AAV9K316Q;
identifying glycosylation site as N57Q, SUMOylation site as K316Q;
predicting Neddylation site K51Q for mutagenesis;
preparing the plasmids using a maxiprep protocol followed by cesium chloride ultracentrifugation; and
confirming the plasmids by restriction digestion and deoxyribonucleic acid (DNA) sequencing.

4. The method for preparing an optimized adeno-associated virus (AAV) vector as claimed in claim 3, wherein synthesizing a microdystrophin transgene, pssAAV-CBA-Kozak-μDys and using pssAAVMHCK7H2-μDys as a transgene control.

5. The method for preparing the optimized AAV vector as claimed in claim 3, further comprising the steps of:
maintaining AAV 293 cells in Iscove's-modified Dulbecco's medium (IMDM) supplemented with 10% FBS, piperacillin and ciprofloxacin;
co-transfecting AAV 293 cells with three plasmids including p.helper, one of AAV9 (rep/cap) wild type and the plasmid mutants AAV9K51Q, AAV9N57Q, and AAV9K316Q, and with the microdystrophin transgene (p.AAV-CBA-kozak-μDys) using polyethelenimine;
replacing medium with complete IMDM 6 hrs post-transfection;
scrapping the cells 72 hrs post-transfection followed by storing at −80° C. till further processing;
lysing the cells followed by digesting with Benzonase to obtain a virus;
purifying the virus by iodixanol gradient ultracentrifugation and ion exchange chromatography; and
determining the virus titers by quantitative PCR using ATCC as standards.

6. The method for preparing the optimized AAV vector as claimed in claim 5, wherein the AAV 293 cells are grown as adherent cultures in incubators maintained at 37° C. and 5% CO₂.

7. The method for preparing the optimized AAV vector as claimed in claim 5, wherein the AAV 293 cells are sub-cultured after treatment with trypsin, washed, and re-suspended in complete medium.

8. The method for preparing the optimized AAV vector as claimed in claim 5, wherein AAV9-WT capsid is configured as a control in packaging the transgene control plasmid p.AAV MHCK7H2-μDys.

9. A composition for gene therapy of muscular dystrophy, comprising the optimized AAV vector as claimed in claim 1, in combination with a pharmaceutically acceptable carrier.

10. The composition for gene therapy of muscular dystrophy as claimed in claim 9, wherein the composition is administered to mdx mice through one of intramuscular route, and intravenous administration at lower vector doses of 1-2×10¹¹ vgs/leg.

11. The composition for gene therapy of muscular dystrophy as claimed in claim 9, wherein the composition is configured for increased microdystrophin expression and restoration of dystrophin glycoprotein complex proteins for improving muscle function.
